(19) United States
(12) Patent Application Publication
(10) Pub. No.: US 2009/0036975 A1
(43) Pub. Date: Feb. 5, 2009

(54) SELF-SENSING STENTS, SMART MATERIALS-BASED STENTS, DRUG DELIVERY SYSTEMS, OTHER MEDICAL DEVICES, AND MEDICAL USES FOR PIEZO-ELECTRIC MATERIALS

(76) Inventors: Kevin Ward, Richmond, VA (US); Zoubeida Ounaies, Richmond, VA (US); George Vetrovec, Richmond, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/097,103

(22) PCT Filed: Dec. 12, 2006
(86) PCT No.: PCT/US2006/061918

§ 371 Date: Sep. 3, 2008

Related U.S. Application Data
(60) Provisional application No. 60/749,092, filed on Dec. 12, 2005.

(51) Int. Cl. A61F 2/06

(52) U.S. Cl. 623/1.18; 623/1.15; 623/1.42

(57) ABSTRACT

A medically implantable stent comprising at least one piezo-electric material may be active, such as by one or more of: delivering an anti-coagulant or other therapeutic effect to a patient in which it is implanted; powering itself; and/or sending an outbound electronic signal to a remote device. When a stent can send such an outbound signal, a physician may non-invasively ascertain the condition of the tissue near the stent.

### FIELD OF THE INVENTION

The invention generally relates to medicine, and especially relates to stents.

### BACKGROUND OF THE INVENTION

Conventionally, stents have been either metallic or polymeric. The stent is inserted to improve flow through an artery by maintaining patency of the artery. Stents are inserted in the artery and expanded to shape by means of an inflatable balloon. Stents are undergoing many changes in designs including the materials from which they are made. Examples include new drug eluting stents which secrete compound from an internal part or luminal surface to prevent clotting. Other designs include incorporating special materials such as NiTl metal, a shape memory alloy (SMA) that exhibits large strains when traversing its transition temperature. The SMA stent can be inserted in the vessel, and as result of experiencing body temperature, enlarges to the required dimensions.

Despite these changes, the complexity of the interface of biomaterials with flowing blood continues to be problematic. Development of clot within the stent resulting in partial or complete occlusion of the stent can still occur. When patients with stents complain of chest pain, the question of the patency of their stent is of great importance. Often, it is necessary to perform coronary angiography on these patients to determine the patency status of the stent.

Stents are now placed in nonvascular structures. These structures now include the airway passages of the respiratory system, the various passages of the urinary system, and the various passages of the gastrointestinal tract.

### SUMMARY OF THE INVENTION

This invention significantly improves upon medical stent technology and provides solutions for challenging problems discussed above. One objective of the present invention is to use new materials and methods which allow interrogation of the stent to determine its patency, especially non-invasive interrogation.

A medically implantable stent comprising at least one piezo-electric material may be advantageously active, such as by one or more of: delivering an anti-coagulant or other therapeutic effect to a patient in which it is implanted; powering itself; and/or sending an outbound electronic signal to a remote device. When a stent can send such an outbound signal, a physician may non-invasively ascertain the condition of the tissue near the stent.

In one preferred embodiment, the invention provides an anti-coagulative and/or antiadhesive stent comprising: a stent implantable into a living patient, the stent comprising at least one negative-charge-producing surface (such as, e.g., a negative-charge-producing surface that comprises at least one piezo-electric material orientation of which is arranged to produce a certain negative charge), and the stent delivering an anticoagulant effect and/or an antiadhesive effect to the patient in which the stent is implanted, such as, e.g., a stent comprising at least one signal-producing component (such as, e.g., a signal-producing component that produces a recordable signal); a stent comprising a piezo-electric material; a drug-eluting stent; a smart stent; a stent comprising a signal-transmitter transmitting an electrical signal a distance in a range of about 1 to 2 feet or more; a stent comprising a recordable voltage output, wherein the recordable voltage output is proportional to at least one function or property of a tissue where the stent is situated (such as, e.g., flow, pressure, force, temperature, etc.); a stent comprising at least one piezo-electric material and a recordable voltage output from interaction between the piezo-electric material and a material in contact with the piezo-electric material; a self-powered stent (such as, e.g., a stent comprising at least one pharmaceutical substance or other substance releasable from the stent and a releasing mechanism for releasing the substance wherein the releasing mechanism is powered by interaction of a piezoelectric material with a tissue in which the stent is situated); a stent comprising PVDF; or copolymers of PVDF with trifluoroethylene (TrFE), tetrafluoroethylene (TFE), PVDF carbon nanotube composites, PVDF nanoclay composites, lead-zirconium-titanate ceramic, or the like; a stent comprising a voltage controller controlling application of voltage (such as controlling application of voltage at a controllable frequency to result in surface vibrations to control interaction of the piezo-electric material with blood (or other fluid) by eliminating blocking or preventing blockage); etc.

The invention in another preferred embodiment provides a method of producing an anticoagulant effect and/or an antiadhesive effect in a patient, comprising a step of: implanting in the patient a stent comprising a negative-charge-producing surface (such as, e.g., a stent comprising a piezo-electric material, a stent comprising PVDF or copolymers of PVDF with trifluoroethylene (TrFE), tetrafluoroethylene (TFE), PVDF carbon nanotube composites, PVDF nanoclay composites, or lead-zirconium-titanate ceramic, and the like; etc.). Examples of the implanting step are, e.g., implanting a drug-eluting stent, implanting a cardiac stent; implanting a coronary artery stent; implanting a vascular stent; implanting an airway stent; implanting a gastrointestinal stent; implanting a urologic stent; implanting a smart stent; etc.

In yet another preferred embodiment, the invention provides a smart stent system, comprising: (a) a smart stent implantable into a living patient, comprising a piezo-electric material and producing a recordable signal; and (b) a signal receiver physically separate from the smart stent and receiving the recordable signal from the smart stent at a distance in a range of about 1 to 2 or more feet, such as, e.g., a smart stent system wherein the receiver is wireless and is coupled either directly or wirelessly to a filter, an amplifier and a monitor (such as, e.g., a computer, a personal digital assistant device, etc.); a smart stent system wherein the smart stent comprises at least one passive component (such as, e.g., a diode bridge to control voltage swings, a voltage regulator, etc.); a smart stent system wherein the smart stent comprises a high density rechargeable battery, a filter, an amplifier, and an A/D converter (microcontroller); a smart stent system wherein an electrical output of the piezoelectric material charges the battery and/or powers at least one component of the stent; a smart stent system comprising a recordable voltage output sent by the stent, wherein the recordable voltage output is proportional to at least one function or property of a tissue where the stent is situated; a smart stent system wherein the stent comprises at least one piezo-electric material, and the system comprises a recordable voltage output from interaction between the piezo-electric material and a material in contact with the piezo-electric material; a smart stent system wherein the stent is self-powered; a smart stent system comprising within the stent at least one substance releasable from the stent, further comprising a releasing mechanism for releasing the substance wherein the releasing mechanism is powered by interaction of a piezoelectric material with a tissue in which the stent is situated; a smart stent system comprising a negative-charge-producing stent; a smart stent system comprising an anticoagulant stent; a smart stent system comprising an antiadhesive stent; a smart stent system comprising a positive-charge producing stent; a smart stent system comprising a procoagulant stent; a smart stent system comprising a proadhesive stent; and other smart stent systems.

The invention also in another preferred embodiment provides a stent comprising a piezo-electric material, the stent being self-powered without needing a separate power source when the stent is implanted in a living patient, such as, e.g., a stent that interferes with undesirable clotting in a patient in which the stent has been implanted; a stent comprising a signal-sender which sends an electronic signal to a location external from the stent, the signal comprising clotting-related information; a stent comprising a recordable voltage output, wherein the recordable voltage output is proportional to at least one function or property of a tissue where the stent is situated; a stent comprising at least one piezo-electric material and a recordable voltage output from interaction between the piezo-electric material and a material in contact with the piezo-electric material; a stent further comprising at least one substance releasable from the stent, and a releasing mechanism for releasing the substance wherein the releasing mechanism is powered by interaction of a piezoelectric material with a tissue in which the stent is situated; a negative-charge-producing stent; an anticoagulant stent; an antiadhesive stent; a positive-charge producing stent; a procoagulant stent; a proadhesive stent; etc.

In another preferred embodiment, the invention provides a method of constructing a smart stent system, comprising the steps of: (a) forming an implantable self-powered stent structure comprising at least one piezo-electric material, and implantable into a patient, the stent structure formed to produce a signal comprising a recordable voltage output proportional to at least one function or property of a tissue where the stent is to be situated; (b) constructing a receiving device that receives the recordable voltage output produced by the stent structure, wherein the receiving device may be separate from the implantable stent structure, such as, e.g., a method further comprising forming in the stent a reservoir for holding a releasable pharmaceutical or other substance and having a release mechanism powered by power generated by the piezo-electric material which power may be used directly or stored or converted before being used to power the release mechanism; a method comprising arranging orientation of the at least one piezo-electric material to produce a certain negative charge or positive charge; and other methods.

The invention also provides, in another preferred embodiment, a drug delivery system, comprising: a container comprising at least one piezo-electric material, the container comprising a cavity for holding an amount of a drug to be released into a patient, wherein the container is implantable in a living patient, such as, e.g., a drug delivery system comprising a drug; a drug delivery system further comprising an electronic signal that is based on an interaction of the piezo-electronic material with a tissue of the patient and that is outbound to a remote device; a drug delivery system comprising a remote control system by which a physician may remotely control at least one parameter relating to release of the drug contained in the container (such as, e.g., number of apertures through which the drug may release; size of apertures through which the drug is released; shape of apertures through which the drug releases; etc.).

In another preferred embodiment the invention provides a monitoring method (such as, e.g., monitoring myocardial function and/or pressure etc., monitoring pulmonary artery function/pressure/flow/temperature, etc., monitoring carotid artery function/pressure/flow, etc., monitoring cerebral artery function/pressure/flow/temperature, etc.) in a patient, comprising: (a) implanting in the patient a stent (such as, e.g., a stent comprising at least one piezo-electric material; a self-powered stent; etc.), (b) receiving from the stent electronic signals relevant to myocardial function/pressure and/or temperature, etc., pulmonary artery function/pressure/flow and/or temperature, etc., carotid artery function/pressure/flow/and/or temperature, etc. or cerebral artery function/pressure/flow and/or temperature, etc. in the patient.

The invention also in another preferred embodiment provides an energy-harvesting device comprising: an energy harvesting structure comprising at least one piezoelectric material situatable in a region of a biologic structure (such as, e.g., a biologic structure within a living patient, a biologic structure within a living organism, etc.); and at least one energy conversion or storage component receiving energy from an interaction of the at least one piezoelectric material with a biologic structure, such as, e.g., an energy-harvesting device comprising a stent; an energy-harvesting device comprising a piezoelectric film; an energy-harvesting device comprising a wrapping wrappable around an artery or other biologic structure; an energy-harvesting device further comprising a monitoring component whereby at least one function (such as, e.g., flow, pressure, temperature, etc.)of the biologic structure is reportable via an electronic signal; an energy-harvesting device wherein function (such as, e.g., flow, pressure, temperature, etc.) of the biologic structure is reported via an electronic signal to a device outside a patient in which the biologic structure is situated; etc.

The invention in another preferred embodiment provides an energy-harvesting method, comprising: situating at least one piezo-electric material on, in or near a biologic structure whereby a piezo-electric interaction occurs in which energy is generated; and converting the energy generated from the piezo-electric interaction into a storable energy and/or an energy useable as power, such as, e.g., energy-harvesting methods are in which the methods further comprise monitoring function (such as, e.g., flow, pressure, temperature, etc.) of the biologic structure (such as, e.g., monitoring function of the biologic structure including sending of electronic signals outside a patient), etc.

### BRIEF DESCRIPTION OF THE DRAWINGS

The foregoing and other objects, aspects and advantages will be better understood from the following detailed description of the preferred embodiments of the invention with reference to the drawings, in which:

FIG. 1 is a block diagram of an embodiment of an inventive stent system. FIG. 1A is a block diagram corresponding to the inventive stent system of FIG. 1 in which there further is provided the capacity for a physician to remotely influence the stent.

FIG. 2 and FIG. 3 are respective circuit diagrams for use in embodiments of inventive stent systems.

FIG. 4 shows a schematic for an experimental setup in Example 2.

FIG. 5 shows a graph for PVDF voltage response, for a sample attached to a simulated artery (using PDMS) in Example 2. The bottom curve is for pressure; the top curve is for voltage.

FIG. 6 shows a graph for PVDF charge response, for the sample of FIG. 5 as in Example 2. The bottom curve is for pressure; the top curve is for charge.

FIG. 7 is graph of average peak to peak voltage versus pressure range, for Example 2. FIG. 7A is table of pressure, pressure range and average peak to peak voltage corresponding to FIG. 7.

FIGS. 8, 8A, 8B are graphs relating to Example 2, of, respectively, pressure range versus average peak to peak charge/area (FIG. 8); pressure range versus average peak to peak charge (FIG. 8A) and pressure range versus average peak to peak current (FIG. 8B).

FIG. 9 is a schematic diagram according to one inventive embodiment of energy-harvesting.

### DETAILED DESCRIPTION OF THE PREFERRED EMBODIMENTS OF THE INVENTION

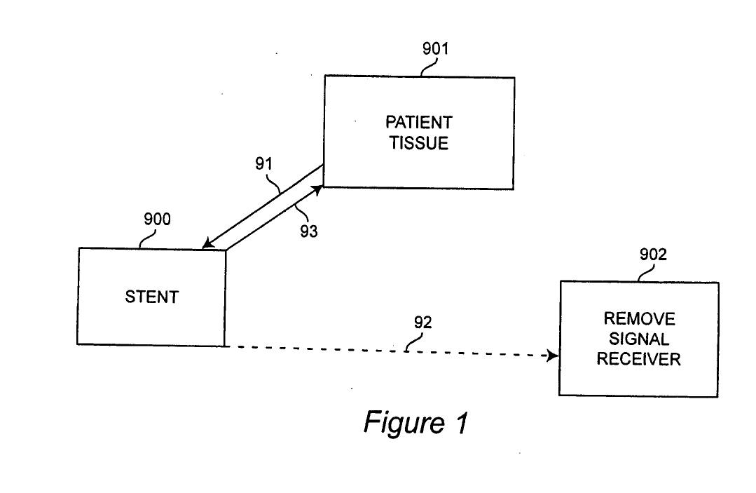

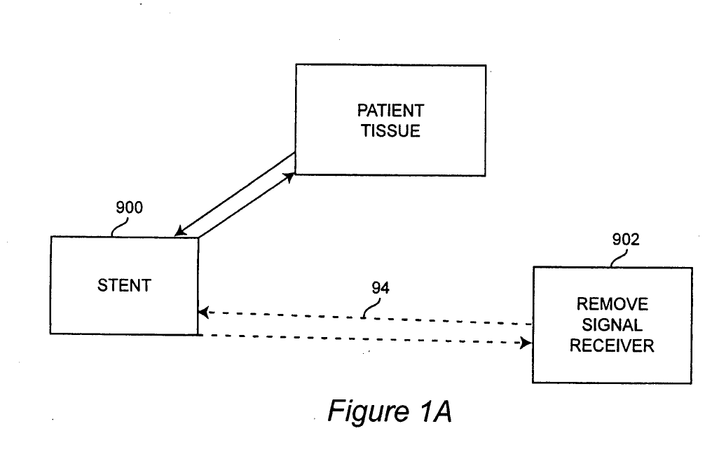

Referring to the figures, beginning with FIG. 1, this invention may be further appreciated, without the invention being limited to the figures. A stent (900) performs at least one, preferably both, of: sending (92) an outbound signal to a remote signal receiver (902), which signal contains information about the tissue (901) in which the stent (900) is implanted, and/or imparting or delivering (93) a therapeutic effect to patient tissue (901). By “remote” we mean that the signal receiver (902) is separate from the stent (900), that is, the receiver (902) need not be implanted in the patient. The remote signal receiver (902) is a particularly advantageous feature of the invention, in that information about an environment (such as, e.g., presence of clotting, etc.) near or within an implanted stent can be appreciated remotely and non-invasively by medical personnel (such as, e.g., a physician).

In FIG. 1, a stent (900) which is implanted in tissue (901) receives an interaction (91) from the patient tissue (901) which interaction (91) may result, advantageously, in one or more of: the stent (900) being able to send an electronic signal (92) to remote signal receiver (902) and/or the stent (900) accumulating storable power or convertible power. The ability of the inventive stent (900) to communicate to a remote signal receiver (902) is a highly advantageous feature of this invention. For example, when a patient has an implanted stent (900), a physician may receive information about the patient's condition in a region of the implanted stent (900) non-invasively.

Electronic signal (92) refers to an electronic or electrical signal such as, e.g., a signal receivable by a wireless receiver such as NanoNet TRX, Crossbow MICA2 Mote, and Microstrain G-link Wireless acceleration system. Most preferably, electronic signal (92) is other than a sound wave.

Preferably a stent (900) is self-powering (excluding battery-powered and the like), which self-powering may be accomplished by constructing the stent (900) using at least one piezo-electric material. Piezoelectricity refers to the ability to convert mechanical energy into electrical energy, or vice versa. Accordingly, piezoelectricity combines both sensing and actuation capabilities. Piezoelectricity arises when permanent dipoles are present and are able to reorient in the presence of an achievable electric field. A piezoelectric stent is capable of converting the energy created by the interaction of the wall of the stent with the blood (or other fluid) flowing past the stent or with the walls of the vessel or other anatomical structure. Examples of a piezo-electric material useable in novel devices are, e.g., piezoelectric polymers (such as, e.g., poly(vinyldene fluoride (PVDF); etc.); piezoelectric polymer composites (such as, e.g., carbon nanotubes-polymer composites; PVDF nanoclay composites; etc.); copolymers of PVDF with trifluoroethylene (TrFE), tetrafluoroethylene (TFF), lead-zirconium-titanate ceramics, etc.

The piezoelectric effect is a linear effect, closely related to the microscopic structure of the material. Piezoelectricity stems from the Greek term “piezo” for “pressure”; it follows that piezoelectricity is the generation of electrical polarization in a material in response to a pressure or mechanical stress. This phenomenon is known as the direct effect. Piezoelectric materials always display the converse effect, where a mechanical deformation ensues upon application of an electrical charge or signal. Piezoelectricity is a property of many non-centrosymmetric (lacking a center of symmetry) ceramics, polymers and other biological systems. The moment-generating nature of this phenomenon makes it ideal for sensors and actuators applications, underlining its similarity to biological-type behavior, where a system senses a change in its environment and reacts to that change through a proportional response.

A subset of piezoelectricity is pyroelectricity. A pyroelectric material is a material that exhibits a change in polarization in response to a uniform temperature variation. Some pyroelectric materials are also ferroelectric; a ferroelectric material possesses a spontaneous polarization that can be reversed by application of a realizable electric field.

We use piezoelectric materials in this invention because those are the only known materials from which energy can be harvested in response to mechanical forces. Therefore by “piezoelectric materials” herein we broadly mean materials from which energy can be harvested, including new materials which might be developed later from which energy can be harvested but which might be called something other than “piezoelectric”.

By varying the piezoelectric parameters, it is possible to increase the sensitivity of the device and enhance monitoring of flow and other parameters. In addition, the piezoelectric nature allows the device to also function as an actuator. An electric field will cause the material to change dimensions along the three axes by contracting or expanding depending on the polarity of the field (longitudinal, transverse and thickness). It is also possible to induce radial deformations in a tube-shaped piezoelectric material. Application of a voltage at a controllable frequency would result in surface vibrations which may control interaction of the piezoelectric material with blood (or other fluid) by eliminating blockage or preventing blockage.

Once the stent is placed in the patient (901), the stent may be interrogated (such as periodically), such as, e.g., in the case of a stent placed in an artery, to ensure that blood is flowing through the artery. Examples of variables that may be targeted are, e.g., flow velocity and pressure. When a physician interrogates a remote signal receiver (902) and learns information about the condition of the patient tissue (901) where the stent (900) is implanted, the physician may, as needed, take action or not according to the physician's judgment.

Referring to FIG. 1, the implanted stent (900) has an interaction (93) with the patient tissue (901) where implanted. When a piezo-electric material is used in the stent (900), advantageously the interaction (93) is managed so that a therapeutic effect is delivered to the patient, such as an anti-coagulant or anti-adhesive effect (such as, e.g., via a negatively-charged surface).

By “anti-coagulative”, we mean the overall inhibition of the formation of a coagulum or clot upon the surfaces of the stent.

By “anti-adhesive”, we mean the overall inhibition of biologic materials such as proteins and cells that might otherwise adhere to the surfaces of a stent.

In some embodiments, optionally, the stent (900) may be equipped as shown in FIG. 1A to receive communication (94) from a remote device (902) such as communication (94) that results from a physician reviewing the results of communication (92) from the stent (900) and determining to adjust the stent (900). That is, the stent (900) optionally may be remotely-adjustible, such as, e.g., remotely adjustable by changing the amount of drug release from the stent (e.g., drug may be contained in small reservoirs) or with release of charge that has been stored from preceding piezoelectric interactions. Such remote changes of a piezo-electric material containing stent (900) are accomplished by, e.g., using the harvested and stored energy to drive various actuators or other MEMS structures which can be remotely activated with an external electrical signal.

Self-powering is preferred for powering stents according to this invention (such as stent (900) in FIGS. 1-1A). Preferably an external or traditional power source (such as battery power) is avoided. For constructing the stent, a material should be used which in contact with living tissue produces a recordable voltage output, such as, e.g., a piezo-electric material or a combination of piezo-electric materials. By using at least one piezo-electric material, a self-powered stent may be constructed, such as a stent whose source of energy comes from the pulsatile flow of blood through the stent. One way, and not the only way, in which a stent may be “smart” is for the stent to comprise a component sending out a recordable voltage output, such as, e.g., a recordable voltage output proportional to at least one function or property of a tissue where the stent is situated. Even in embodiments in which a battery becomes part of the stent, advantageously this battery is charged by the electrical output produced by the piezoelectric material and thus inductive charging or recharging of the battery is not necessary.

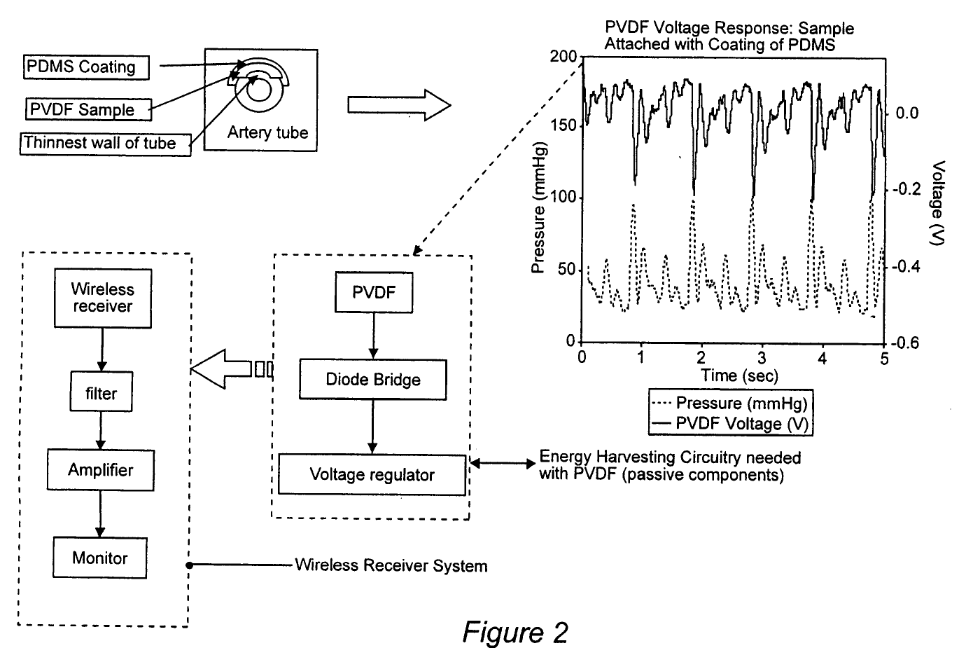

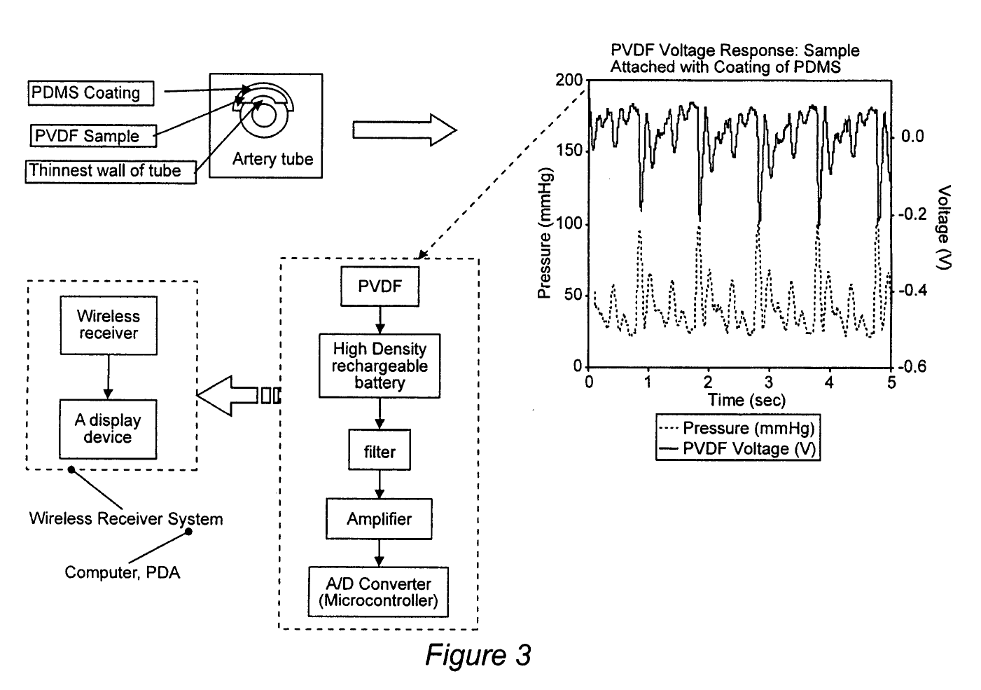

For examples of converting pulsatile flow of the blood through a stent into an electronic signal which may be output to a receiver device and of converting pulsatile flow of the blood through a stent into storable power, see, e.g., the circuitry in FIGS. 2 and 3.

Examples of a signal-producing component that may be used in an inventive stent are, e.g., a signal-producing component that produces a recordable signal; a signal-transmitter transmitting an electrical signal (such as a transmitter transmitting an electrical signal in a range of about 1-2 feet or greater).

Non-limiting examples of circuitry for a receiver (902) are shown in FIGS. 2 and 3 which depict alternative wireless receiver systems. Examples of a wireless receiver are, e.g., NanoNet TRX, Crossbow MICA2 Mote, and Microstrain G-link Wireless acceleration system. In FIG. 2, the monitor may be, for example, a computer, a PDA, etc.

In FIG. 2, a voltage regulator is shown, but it may not be needed.

In the circuitry of FIG. 3, a high density rechargeable battery, such as the ones used in pacemakers, is used. However, the battery is charged via the power created the piezo electric material in the stent.

A stent according to this invention may be implanted in a living patient in a place such as a place, e.g., where a physical stent structure is deemed to be advantageous to the patient (such as, e.g., a clogged artery, etc.); where delivery of a pharmaceutical substance or other substance is wanted to be accomplished; where monitoring of clotting is desired; where monitoring of at least one function or property (such as, e.g., flow, pressure, temperature, etc.) of a tissue is wanted; etc. Examples of where a stent in this invention may be situated are, e.g., in a path of blood flow, in airway flow, urine flow, bile flow, etc.

When an inventive stent comprising a piezoelectric material is placed in blood flow, usually generating an outbound signal should be possible. However, in some embodiments of the invention, such as, e.g., when using a stent comprising a piezoelectric material in airway flow or urine flow, it may not be possible to generate an outbound signal but the stent still would be providing the negative charge having a therapeutic effect.

In practicing the invention, a drug is not required to be eluted from the stent but advantageously the invention provides that a drug optionally may be eluted from a smart stent. A drug that may be eluted from the inventive stent is not particularly limited and examples are, e.g., antiproliferative and anti-inflammatory agents.

The present invention may be used in various contexts and ways, such as, for example, in conventional applications for stents (including drug-eluting stents and non-drug-eluting stents) where adverse consequences can now better be avoided.

However, the shape of an article used in the present invention is not particularly limited, and may include shapes and sizes conventionally used for stents, as well as shapes not previously associated with stents.

The present invention may also be used in applications (such as drug-delivery applications, structural applications, etc.) where previously stents may not have yet been used conventionally. Especially in such drug-delivery applications an inventive article or device is not required to have a traditional stent shape and an inventive device comprising a piezo electric material may be various shapes.

Stents according to this invention may be used to overcome the clotting difficulties that have been identified with conventional stents inserted into arteries through which blood flows. The novel stents in many instances prevent clot formation and thereby avoid the clotting problem, and, in cases where clot formation cannot be avoided, at least the fact of clot formation will be made known, so that appropriate action can be taken.

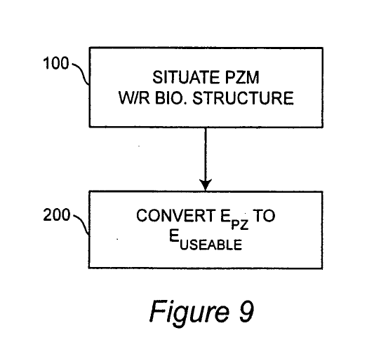

The present inventors also provide for the inventive use of piezo-electric materials in harvesting energy from a biologic structure or based on functioning of a biologic structure, by situating (100) at least one piezo-electric material with respect to a biologic structure (FIG. 9), whereby energy (E_{PZ}) is generated by interaction of the piezo-electric material and the biologic structure. The situating (100) may be, e.g., by implanting in a living patient, such as by, e.g., inserting a stent comprising at least one piezo-electric material; wrapping an artery with a film or wrap comprising at least one piezo-electric material; etc. The piezo-electric material may be situated in, on or near the biologic structure, and in different embodiments may directly or indirectly contact the biologic structure. In this setting the piezo-electric material is harvesting energy from its surrounding which may be used to monitor or control the surroundings.

The step of situating the piezo-electric material with respect to the biologic structure is followed by a step of converting (200) the energy (E_{PZ}) generated by the piezo-electric interaction to energy (E_{useable}) useable for powering an application or storable for later use powering an application, such as energy useable for powering an antenna, energy useable for powering circuitry, etc.

The invention may be appreciated with reference to the following examples, without the invention being limited to the examples.

### EXAMPLE 1

In this example, “smart” biomaterials are used that allow their implantation and measurement of intravascular flow and pressure. This stent design is based on piezoelectric materials. Sensing capabilities are incorporated either intrinsic to the stent or coupled to the stent.

Once the stent of this example is placed in a patient, it is interrogated periodically to ensure that blood is flowing through the artery. Flow velocity and pressure are two variables that are targeted. This interrogation is accomplished remotely. An antenna is integrated with the device to take the sensor signal and transfer the signal into an electromagnetic signal, which is then transmitted outside the body and picked up remotely.

Based on these principles, it is possible to monitor other aspects of myocardial function from the stent. This includes but is not limited to contractility parameters from the coronary artery-myocardial surface.

Because some stents are placed angiographically using left heart catheterization, piezoelectric materials/devices may be attached to the ascending aorta to measure aortic pressure and flow (cardiac output) or within the left ventricle to measure intraventricular pressure. The same may be done in the right heart using pulmonary artery catheterization. These stents and materials also may be used for other vessels including but not limited to the carotid and cerebral arteries.

Features and advantages of this invention including unique features and advantages, are as follows:
A novel device that results in dual sensing and actuation functions is provided. These functions can either be present in the same device or be in close proximity such as in a layered configuration.
A novel stent design that permits internal sensing designed to monitor cardiovascular and respiratory phenomena is provided.
A novel stent design based on piezoelectric polymers and polymer composites which typically display high voltage response, wide frequency bandwidth and flat frequency response is provided.
The stent design allows continuous monitoring to detect blockage or other changes in blood dynamics.
The device can be miniaturized and can incorporate the sensing and actuation functions as well as the circuitry needed to activate or interrogate it remotely.
The piezoelectric polymers and polymer composites are flexible, and can conform to various shapes such as tubes. They have densities similar to water and living tissues, and are chemically inert, which makes them suitable for implantation.
The piezoelectric polymers and polymer composites have good impedance matching to water and living tissue.
The materials for use in the stents are flexible and lightweight, therefore they will not result in patient discomfort nor will they interfere with the regular function of arteries and other vessels.
Transduction process in piezoelectric polymers and polymer composites is frequency- and temperature-responsive, and covers a broad dynamic range.
The novel device makes possible energy harvesting potential for self-powering or to power remote functions.
The piezoelectric materials are compatible with MEMS and nanodevice processing.
The novel device makes possible application of a voltage at a controllable frequency, which would result in surface vibrations which may control interaction of the piezoelectric material with blood (or other fluid) by eliminating blockage or preventing blockage.
General microamp- to nanoamp- or microcoulomb to nanacoulomb level negative current is extremely advantageous because clogging of the stent may be inhibited. In fact, it has been shown that a negative charge is extremely antithrombotic/anticoagulative/antiadhesive and thus might prevent restenosis if the purpose of the implanted stent it to restore and maintain blood flow to tissue. Orientation of the dipoles of the piezoelectric material in relationship to forces applied to it provide for the potential to produce a negative current or charge in response to straining a piezoelectric material. Orientation of the dipoles is accomplished by known means such as application of a strong DC electrical field to the piezoelectric materials which align the dipoles parallel to the electric field. Increasing or decreasing pressure parallel to the dipoles on a piezoelectric material causes electrical charge to be generated which can be positive or negative depending on the piezoelectric coefficients of the particular piezoelectric material. In addition to this charge existing at the surface of the stent, this charge can in turn flow and/or be stored if desired using necessary electrical components to perform certain functions. Piezoelectricity being a linear phenomenon that relates an applied mechanical stimulus to a resulting electrical charge or current, it follows that the magnitude of electrical charge or current produced would increase or decrease depending on whether the magnitude of the pressure is increasing or decreasing. Depending on the piezoelectric coefficients of the material if the pressure on the piezoelectric material is parallel to the initial polarization, either a positive or negative electrical potential is generated. A number of options are available to sustain continuous, small amplitude, negative current, such as, e.g.:
(1) biasing the piezoelectric stent such that the additional stimulus will result in a current oscillating in the negative domain. The result will be a negative current with an amplitude that reaches a minimum and a maximum, following the frequency of the external stimulus.
(2) incorporating a diode bridge into the stent to prevent fluctuations of current into the positive range.
(3) using signal conditioning to invert the sign of the current when it becomes positive.
(4) storing the current harvested from the piezoelectric material, and releasing it after an AC to DC conversion. The resulting DC current is then inverted if needed to yield a continuously negative current.

The invention can be used for implanted intravascular monitoring of blood flood and pressure. Furthermore, polymers and polymer composites have high sensitivity, which means they are efficient voltage generators in response to mechanical stimulus. When implanted, the sensor device transforms the mechanical energy due to blood flow, breathing or other physiologic functions into charge. The charge is stored and used to power up other implanted devices. A 30 μm thin piezoelectric polymer, 100 cm long and 2 cm wide, rolled around the thorax of a patient breathing at the rate of 24/min, can create power as high as 500 μW [Hausler et al. 1980, Proceedings of IEEE].

### EXAMPLE 2

In this Example, research was focused on the use of PVDF and its ability to provide a link between mechanical stimulus and electrical output. PVDF refers to Poly(vinylidene fluoride), which is a commercially available polymer, demonstrates piezoelectricity, has high resistance to both heat and electricity, and is highly non-reactive. Previous application of PVDF include: electrical and chemical insulators, speakers, strain gauges, voltage sources, and various sensor applications.

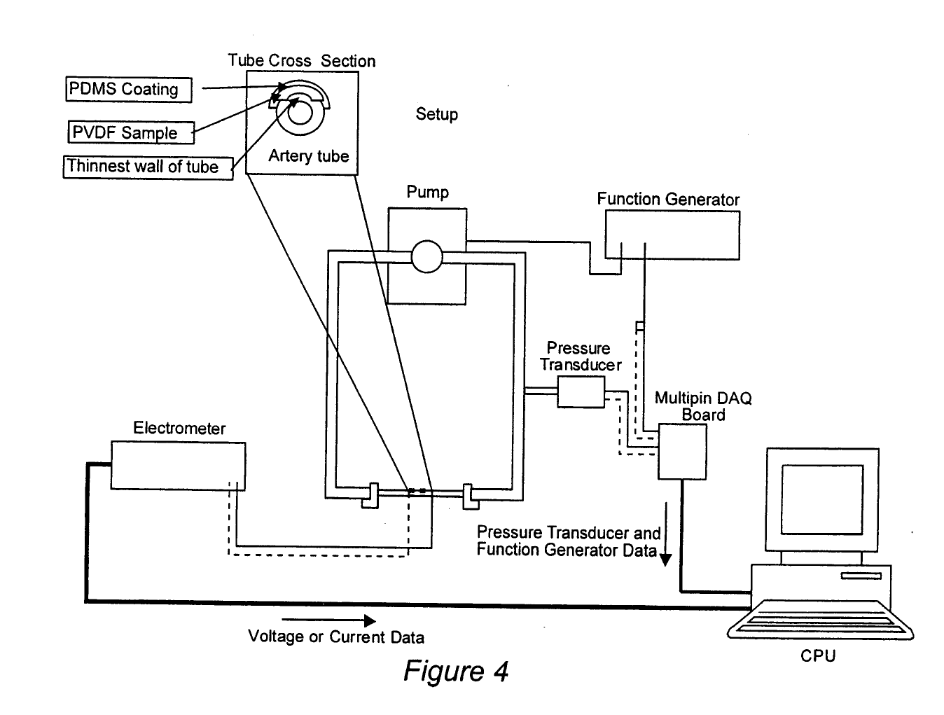

An experimental setup was established according to FIG. 4.

The following test was performed to test the effect of the flow pressure range on the voltage response of the PVDF. The test was performed using PVDF in a coated configuration at a frequency of 1 Hz. Data was taken at each range and the average of the peak to peak voltage of 5 cycles was calculated.

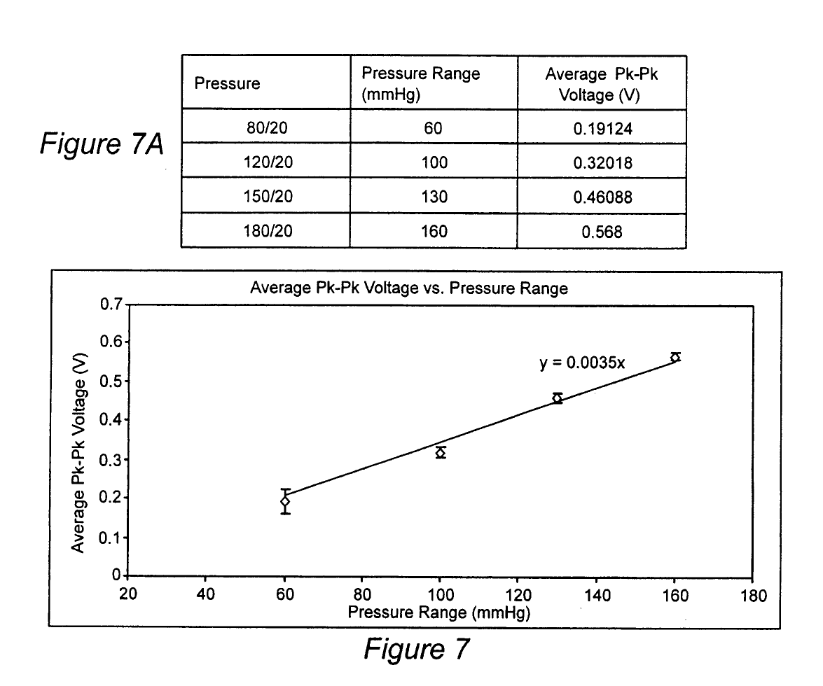

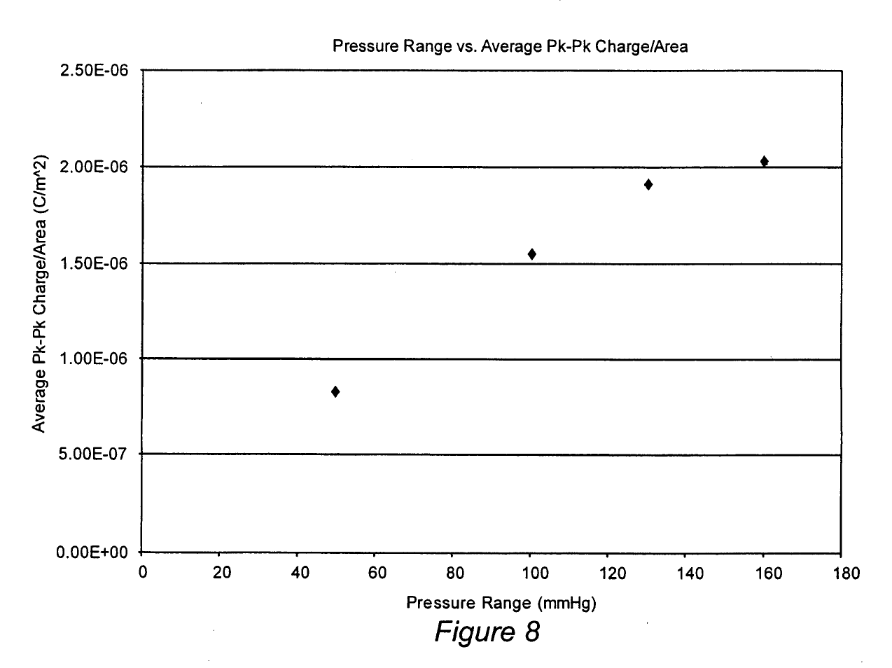

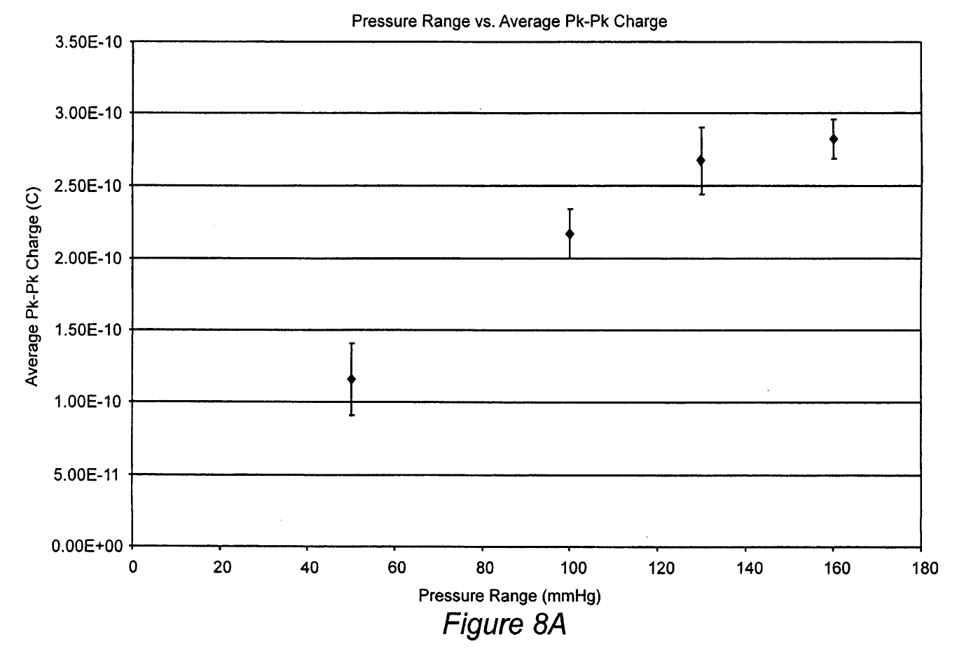

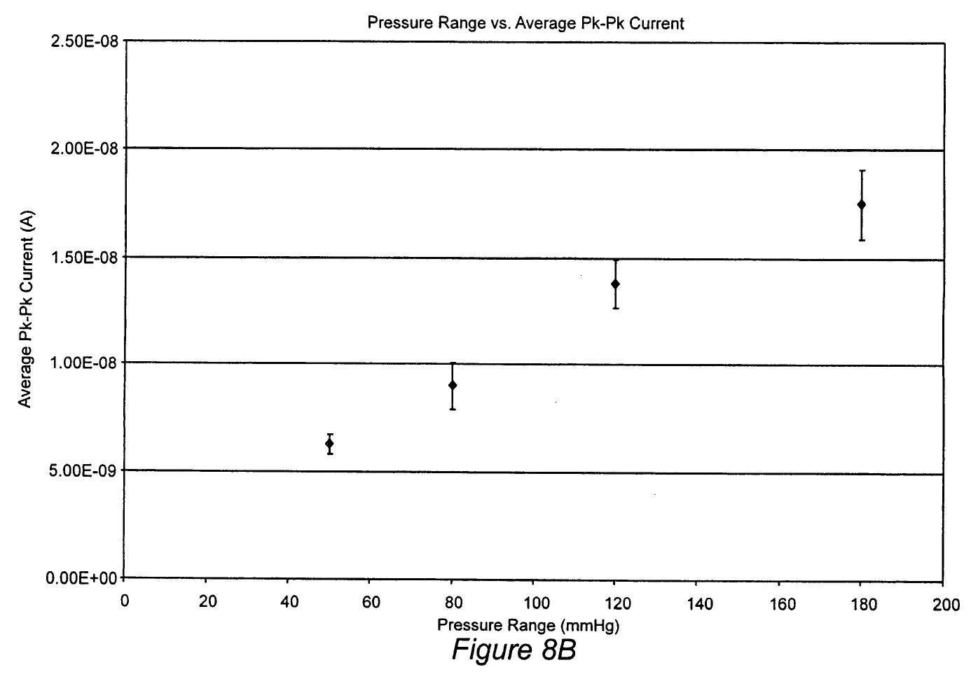

The results (see FIGS. 7-8B) show that an increase in the pressure range brings about an increase in the PVDF voltage response. Furthermore, the data demonstrate that a negative charge is produced and this response is also linear. The relationship is linear, as expected for a piezoelectric-driven response.

For FIGS. 8, 8A, 8B, the tests were performed using the same setup (FIG. 10) and sample as the test done to attain pressure range versus peak to peak voltage. For FIG. 8, charge was measured and the divided by the electrode area of the sample (7×20 mm).

The experimental data of this Example 2 show that a PVDF material (which is a commercially available piezo-electric material) when used with a simulated biologic structure (in this example, PDMS material used to simulate an artery) demonstrates experimental results from which to conclude that when a stent comprising such materials is implanted in a patient, such a stent would be expected to self-power, exhibit anti-coagulant behavior, exhibit anti-adhesive effect, and when fitted with an antenna be capable of sending electronic signals to a receiver not implanted in the patient.

### EXAMPLE 3 (SELF-POWERED STENT)

A piezo-electric material-containing stent is fitted with energy conversion and/or energy storage components by which piezo-electrically obtained energy (i.e., energy obtained via the piezo-electrical interaction with the tissue in which the product will be implanted) is converted to electrical signals and/or stored. Examples of energy conversion and energy storage components are, e.g., capacitors, batteries, diodes, transformers, etc.

Circuitry is included for using the stored energy to power one or more energy-using components (such as, e.g., a releasing mechanism on a drug-containing reservoir; an antenna; etc.) contained in, on, contiguous, near or separate from the stent.

### EXAMPLE 4

In this inventive example, a stent is used to power itself or components/systems that are contiguous or separate from it. For example, thus a stent in a cerebral or coronary artery (or the aorta) is used to harvest energy from those sites which is in turn used to power devices (or charge batteries) which may be short distances away from it.

### EXAMPLE 5

In this inventive example, piezoelectric materials are wrapped around arteries or other biologic structures to harvest energy and direct the energy to other sources or to use the energy for monitoring the structure to which the piezoelectric materials are attached.

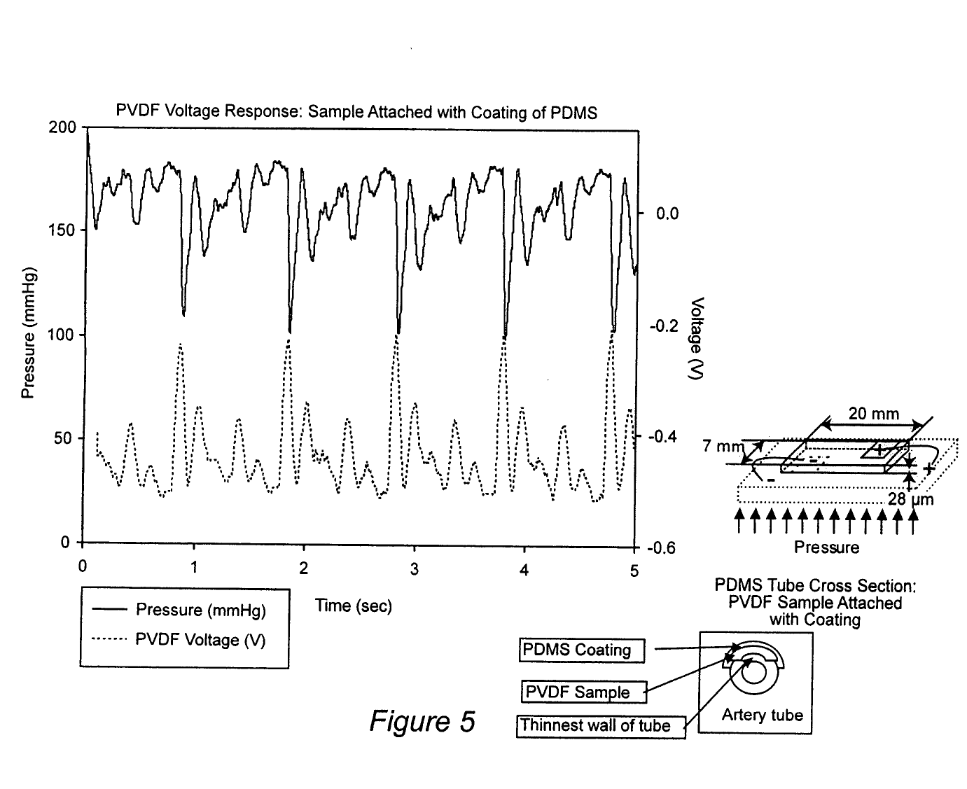

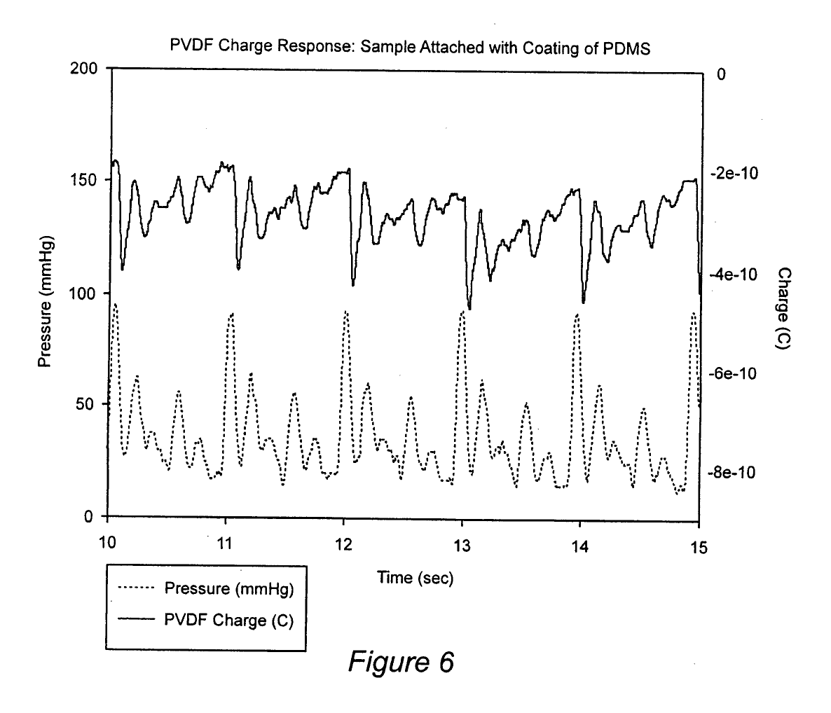

Our experimental data (see Example 2 and FIGS. 4-8B) show that a recordable signal was produced by PVDF materials (i.e., a piezo-electrical material) wrapped around tubing (which mimics the artery). Despite the piezo-electric materials not having direct contact with the lumen of the tubing, a signal can still be recorded. Therefore, the invention provides extraluminal uses as well as intraluminal uses.

Thus for surgeries such as coronary artery bypass grafting, arterial bypass surgeries of the leg, or carotid endarterectormies, the surgeon could wrap a piezoelectric film around the vessel which could serve the purpose of monitoring blood flow or other function. The negative charge produced in this case would not be helpful to the lumen of the vessel, but the sensing function would still maintain its usefulness. The material in this case is not being used to stent the vessel open but instead to monitor the vessel.

While the invention has been described in terms of its preferred embodiments, those skilled in the art will recognize that the invention can be practiced with modification within the spirit and scope of the appended claims.

We claim:

1. An anti-coagulative and/or antiadhesive stent comprising: a stent implantable into a living patient, the stent comprising at least one negative-charge-producing surface, and the stent delivering an anticoagulant effect and/or an antiadhesive effect to the patient in which the stent is implanted.

2. The stent of claim 1, comprising at least one signal-producing component.

3. The stent of claim 2, wherein the signal-producing component produces a recordable signal.

4. The stent of claim 1, comprising a piezo-electric material.

5. The stent of claim 1, wherein the at least one negative-charge-producing surface comprises at least one piezo-electric material orientation of which is arranged to produce a certain negative charge.

6. The stent of claim 1, comprising a drug-eluting stent.

7. The stent of claim 1, comprising a smart stent.

8. The stent of claim 1, comprising a signal-transmitter transmitting an electrical signal a distance in a range of about 1 to 2 feet or more.

9. The stent of claim 1, comprising a recordable voltage output, wherein the recordable voltage output is proportional to at least one function or property of a tissue where the stent is situated.

10. The stent of claim 9, wherein the at least one function or property is selected from the group consisting of: flow, pressure, force, and temperature.

11. The stent of claim 1, comprising at least one piezo-electric material and a recordable voltage output from interaction between the piezo-electric material and a material in contact with the piezo-electric material.

12. The stent of claim 1, comprising a self-powered stent.

13. The stent of claim 12, comprising at least one pharmaceutical substance or other substance releasable from the stent and a releasing mechanism for releasing the substance wherein the releasing mechanism is powered by interaction of a piezoelectric material with a tissue in which the stent is situated.

14. The stent of claim 1, comprising at least one selected from the group consisting of PVDF; copolymers of PVDF with trifluoroethylene (TrFE), tetrafluoroethylene (TFE), PVDF carbon nanotube composites, PVDF nanoclay composites, lead-zirconium-titanate ceramic.

15. The stent of claim 1, comprising a voltage controller controlling application of voltage.

16. A method of producing an anticoagulant effect and/or an antiadhesive effect in a patient, comprising a step of: implanting in the patient a stent comprising a negative-charge-producing surface.

17. The method of claim 16, wherein the stent comprises a piezo-electric material.

18. The method of claim 16, wherein the implanting step comprises implanting a drug-eluting stent.

19. The method of claim 16, wherein the implanting step comprises implanting a stent selected from the group consisting of a cardiac stent; a coronary artery stent; a vascular stent; an airway stent; a gastrointestinal stent; and a urologic stent.

20. The method of claim 16, wherein the implanting step comprises implanting a smart stent.

21. The method of claim 16, wherein the stent comprises at least one selected from the group consisting of PVDF; copolymers of PVDF with trifluoroethylene (TrFE), tetrafluoroethylene (TFE), PVDF carbon nanotube composites, PVDF nanoclay composites, and lead-zirconium-titanate ceramic.

22. A smart stent system, comprising: (a) a smart stent implantable into a living patient, comprising a piezo-electric material and producing a recordable signal; and (b) a signal receiver physically separate from the smart stent and receiving the recordable signal from the smart stent at a distance in a range of about 1 to 2 or more feet.

23. The smart stent system of claim 21, wherein the receiver is wireless and is coupled either directly or wirelessly to a filter, an amplifier and a monitor.

24. The smart stent system of claim 22, where in the monitor is selected from the group consisting of a computer and a person al digital assistant device.

25. The smart stent system of claim 22, wherein the smart stent comprises at least one passive component selected from the group consisting of: a diode bridge to control voltage swings and a voltage regulator.

26. The smart stent system of claim 22, wherein the smart stent comprises a high density rechargeable battery, a filter, an amplifier, and an A/D converter (microcontroller).

27. The smart stent system of claim 22, wherein an electrical output of the piezoelectric material charges the battery and/or powers at least one component of the stent.

28. The smart stent system of claim 22, comprising a recordable voltage output sent by the stent, wherein the recordable voltage output is proportional to at least one function or property of a tissue where the stent is situated.

29. The smart stent system of claim 22, wherein the stent comprises at least one piezo-electric material, and the system comprises a recordable voltage output from interaction between the piezo-electric material and a material in contact with the piezo-electric material.

30. The smart stent system of claim 22, wherein the stent is self-powered.

31. The smart stent system of claim 22, comprising within the stent at least one substance releasable from the stent, further comprising a releasing mechanism for releasing the substance wherein the releasing mechanism is powered by interaction of a piezoelectric material with a tissue in which the stent is situated.

32. The smart stent system of claim 22, comprising a stent selected from the group consisting of: a negative-charge-producing stent; an anticoagulant stent; an antiadhesive stent; a positive-charge producing stent; a positive-charge-producing stent; a procoagulant stent; and a proadhesive stent.

33. A stent comprising: a piezo-electric material, the stent being self-powered without needing a separate power source when the stent is implanted in a living patient.

34. The stent of claim 33, wherein the stent interferes with undesirable clotting in a patient in which the stent has been implanted.

35. The stent of claim 33, comprising a signal-sender which sends an electronic signal to a location external from the stent, the signal comprising clotting-related information.

36. The stent of claim 33, comprising a recordable voltage output, wherein the recordable voltage output is proportional to at least one function or property of a tissue where the stent is situated.

37. The stent of claim 33, comprising at least one piezo-electric material and a recordable voltage output from interaction between the piezo-electric material and a material in contact with the piezo-electric material.

38. The stent of claim 33, further comprising at least one substance releasable from the stent, and a releasing mechanism for releasing the substance wherein the releasing mechanism is powered by interaction of a piezoelectric material with a tissue in which the stent is situated.

39. The stent of claim 33, comprising a stent selected from the group consisting of: a negative-charge-producing stent; an anticoagulant stent; an antiadhesive stent; a positive-charge producing stent; a positive-charge-producing stent; a procoagulant stent; and a proadhesive stent.

40. A method of constructing a smart stent system, comprising the steps of: (a) forming an implantable self-powered stent structure comprising at least one piezo-electric material, and implantable into a patient, the stent structure formed to produce a signal comprising a recordable voltage output proportional to at least one function or property of a tissue where the stent is to be situated; (b) constructing a receiving device that receives the recordable voltage output produced by the stent structure, wherein the receiving device may be separate from the implantable stent structure.

41. The method of claim 40, further comprising: forming in the stent a reservoir for holding a releasable pharmaceutical or other substance and having a release mechanism powered by power generated by the piezo-electric material which power may be used directly or stored or converted before being used to power the release mechanism.

42. The method of claim 40, comprising: arranging orientation of the at least one piezo-electric material to produce a certain negative charge or positive charge.

43. A drug delivery system, comprising: a container comprising at least one piezo-electric material, the container comprising a cavity for holding an amount of a drug to be released into a patient, wherein the container is implantable in a living patient.

44. The drug delivery system of claim 43, comprising a drug.

45. The drug delivery system of claim 43, further comprising an electronic signal that is based on an interaction of the piezo-electronic material with a tissue of the patient and that is outbound to a remote device.

46. The drug delivery system of claim 43, comprising a remote control system by which a physician may remotely control at least one parameter relating to release of the drug contained in the container.

47. The drug delivery system of claim 46, wherein the parameter controlled is selected from the group consisting of: number of apertures through which the drug may release; size of apertures through which the drug is released; shape of apertures through which the drug releases.

48. A method of myocardial monitoring, pulmonary artery monitoring, carotid artery monitoring, or cerebral artery monitoring in a patient, comprising: (a) implanting in the patient a stent, (b) receiving from the stent electronic signals.

49. The method of claim 48, wherein the receiving step comprises receiving from the stent electronic signals relevant to one selected from the group consisting of: myocardial function, myocardial pressure, myocardial temperature, pulmonary artery function, pulmonary artery pressure, pulmonary artery flow, pulmonary artery temperature, carotid artery function, carotid artery pressure, carotid artery flow, cerebral artery function, cerebral artery pressure, cerebral artery flow and cerebral artery temperature in the patient.

50. The method of claim 49, wherein the stent comprises at least one piezo-electric material.

51. The method of claim 49, wherein the stent is self-powered.

52. An energy-harvesting device comprising: an energy harvesting structure comprising at least one piezoelectric material situatable in a region of a biologic structure; and at least one energy conversion or storage component receiving energy from an interaction of the at least one piezoelectric material with a biologic structure.

53. The energy-harvesting device of claim 53, comprising a stent.

54. The energy-harvesting device of claim 53, comprising a piezoelectric film.

55. The energy-harvesting device of claim 53, comprising a wrapping wrappable around an artery or other biologic structure.

56. The energy-harvesting device of claim 53, wherein the biologic structure is within a living patient.

57. The energy-harvesting device of claim 53, further comprising a monitoring component whereby at least one function of the biologic structure is reportable via an electronic signal.

58. The energy-harvesting device of claim 53, wherein at least one selected from the group consisting of flow, pressure and temperature of the biologic structure is reported via an electronic signal to a device outside a patient in which the biologic structure is situated.

59. An energy-harvesting method, comprising: situating at least one piezo-electric material on, in or near a biologic structure whereby a piezo-electric interaction occurs in which energy is generated; and converting the energy generated from the piezo-electric interaction into a storable energy and/or an energy useable as power.
